(19) United States
(12) Patent Application Publication
(10) Pub. No.: US 2013/0211482 A1
(43) Pub. Date: Aug. 15, 2013

(54) THERAPEUTIC APPARATUS, COMPUTER-IMPLEMENTED METHOD, AND COMPUTER PROGRAM PRODUCT FOR CONTROLLING THE FOCUS OF RADIATION INTO A MOVING TARGET ZONE

(71) Applicant: Kari Vaino, Tapio Piipponen, Vantaa (FI)

(72) Inventor: Kari Vaino, Tapio Piipponen, Vantaa (FI)

(73) Assignee: KONINKLIJKE PHILIPS ELECTRONICS N.V., EINDHOVEN (NL)

(21) Appl. No.: 13/881,027

(22) PCT Filed: Oct. 25, 2011
(86) PCT No.: PCT/IB2011/054757

§ 371 Date: Apr. 23, 2013

(30) Foreign Application Priority Data
Oct. 26, 2010 (EP) 10188817.0

(51) Int. Cl. A61N 5/01

(52) U.S. Cl. CPC A61N 5/01
USPC 607/100

(57) ABSTRACT

A therapeutic apparatus (300, 400, 500, 600) comprising: a therapeutic system (302, 402) for treating a target zone (304) of a subject (306), wherein the therapeutic system has an adjustable focus for directing radiation (410) into the target zone; a respiration sensor (308, 310, 416, 424) for measuring a respiratory phase of the subject; a processor (322) for controlling the therapeutic apparatus; and a memory (326, 328) containing machine executable instructions (340, 342, 344, 346, 348, 350, 352) for execution by the processor, wherein execution of the instructions causes the processor to: send (100, 206) control signals to the therapeutic system that cause treatment of the target zone, receive (102, 208) time-dependant-respiratory-phase-data (330) from the respiration sensor, generate (104, 210) focus-adjustment-control-signals in accordance with the time-dependant-respiratory-phase-data, and send (106, 212) the focus-adjustment-control-signals to the therapeutic system.

### TECHNICAL FIELD

The invention relates to the control of a therapeutic apparatus for focusing radiation into a focus of a target zone, in particular to the control of the focus using a respiratory sensor.

### BACKGROUND OF THE INVENTION

Effective treatment of moving tumors using radiation therapy or High-intensity focused ultrasound (HIFU) requires real-time three-dimensional information of the location of the target. This information is needed to guarantee sufficient dose for the target and to avoid dosing the surrounding healthy tissue. The movement of a tumor is typical in abdominal organs such as the pancreas, the liver and the kidney. The movement is periodic by nature and it is due to breathing of the patient. The displacement of a tumor within the respiratory cycle can be up to few centimeters which is large in comparison with the size of the HIFU focal zone.

Respiratory gating is a method used in radiation therapy to compensate respiratory movement: The breathing signal is measured, then, volumetric images are taken with the CT scanner in different phases of the respiratory cycle to model the movement of the target. During treatment, the breathing signal is also measured and the therapeutic beam is switched of whenever the target is outside the predefined window. Other respiratory motion detection methods include: spirometry, tracking the position of an external marker placed on the skin of the patient, a belt with a strain gauge, stereophonic imaging of the torso, and time-of-flight imaging of the torso

US patent application publication US 2010/0094153 A1 discloses a respiration sensor that is adapted to extend from a first point of contact on one side of a patient to a second point of contact on an opposing side of the patient. Respiratory signal data is analyzed in real time and is used to gate a CT scanner thereby creating images that are phase-coordinated.

### SUMMARY OF THE INVENTION

The invention provides for a therapeutic apparatus, a computer-implemented method, and a computer program product in the independent claims. Embodiments are given in the dependent claims.

A difficulty with existing therapeutic apparatuses is that the therapy or the acquisition of medical imaging data is gated according to the phase of the respiratory cycle. Some embodiments of the invention adjust the focus of a therapeutic system based on the respiratory phase of the subject. Some embodiments use a model which predicts the location of the focus based on the respiratory phase of the subject.

Some embodiments use a force sensor integrated into a subject support as a respiratory sensor. The use of such a therapeutic apparatus may have the following benefits:
1. Patient convenience is improved. No disturbing respiration measurement apparatus or belt is connected to the patient.
2. Product integration level is increased. There are fewer accessories related to the treatment. The respiratory measurement device is an integral part of the table top. It is seamless to the patient and to the nurse.
3. The respiratory measurement device has native support from the HIFU platform. This may have the benefit that no interface to the imaging software is needed.

Some embodiments combine a force sensor which measures a ballistocardiogram signal (BCG), respiration triggered magnetic resonance imaging, and Magnetic Resonance-guided (MR-guided) High Intensity Focused Ultrasound (HIFU) with focal point adaption to a defined HIFU treatment session for tumors moving due to patient respiration. It has three steps.

First, the BCG (the Ballistocardiogram) signal is measured with a force sensor located to the table top. The BCG is used to detect respiratory movement of the patient. The BCG signal consists of the force applied to the table top due to heart beat and breathing of the patient. To measure the respiratory motion a very sensitive force sensor is needed and the output signal is low-pass filtered.

Second, the measured breathing signal is used as trigger for volume images of the organ under interest in the pre-treatment phase (for example MRI can be used). Volume images may be taken in different phases of the respiratory cycle. The result is a sequence of volume images, each representing different phase (θ) of the respiratory cycle. The respiratory signal may be measured with conventional methods. In the some embodiments of the invention, BCG triggered MRI is used. Next, a respiratory phase dependent periodic vector which is a function of θ is assigned to the position of the target. The vector pointing to the target can be calculated, and used to control the targeting of the focus.

Finally, the breathing signal is further measured in real-time during the treatment phase. Now, a periodic vector provides target location information for each respiratory phase. This real-time target location information is used as input for the phased array transducer control electronics to move the focal point along with the moving tumor in MR-guided HIFU.

The force sensor may be located to the table top in such a way that it supports a portion or all of the subject's weight. The output signal is the BCG signal and after low-pass filtering it represents the patient breathing signal. The sensor can be used as an optional accessory or it can be an integral part of the table top.

A ‘computer-readable storage medium’ as used herein encompasses any tangible storage medium which may store instructions which are executable by a processor of a computing device. The computer-readable storage medium may be referred to as a computer-readable non-transitory storage medium. The computer-readable storage medium may also be referred to as a tangible computer readable medium. In some embodiments, a computer-readable storage medium may also be able to store data which is able to be accessed by the processor of the computing device. Examples of computer-readable storage media include, but are not limited to: a floppy disk, a magnetic hard disk drive, a solid state hard disk, flash memory, a USB thumb drive, Random Access Memory (RAM) memory, Read Only Memory (ROM) memory, an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example a data may be retrieved over a modem, over the internet, or over a local area network.

Computer memory is an example of a computer-readable storage medium. Computer memory is any memory which is directly accessible to a processor. Examples of computer memory include, but are not limited to: RAM memory, registers, and register files.

Computer storage is an example of a computer-readable storage medium. Computer storage is any non-volatile computer-readable storage medium. Examples of computer storage include, but are not limited to: a hard disk drive, a USB thumb drive, a floppy drive, a smart card, a DVD, a CD-ROM, and a solid state hard drive. In some embodiments computer storage may also be computer memory or vice versa.

A processor is an electronic component which is able to execute a program or machine executable instruction. References to the computing device comprising “a processor” should be interpreted as possibly containing more than one processor. The term computing device should also be interpreted to possibly refer to a collection or network of computing devices or computers each comprising a processor. Many programs have their instructions performed by multiple processors that may be within the same computing device or which may even distributed across multiple computing device.

A ‘user interface’ as used herein is an interface which allows a user or operator to interact with a computer or computer system. A user interface may provide information or data to the operator and/or receive information or data from the operator. The display of data or information on a display or a graphical user interface is an example of providing information to an operator. The receiving of data through a keyboard, mouse, trackball, touchpad, pointing stick, graphics tablet, joystick, gamepad, webcam, headset, gear sticks, steering wheel, pedals, wired glove, dance pad, remote control, and accelerometer are all examples of receiving information or data from an operator.

‘Medical image data’ as used herein encompasses two or three dimensional data that has been acquired using a medical imaging system.

A ‘medical imaging system’ as used herein encompasses an apparatus adapted for acquiring information about the physical structure of a patient and construct sets of two dimensional or three dimensional medical image data. Medical image data can be used to construct visualizations which are useful for diagnosis by a physician. This visualization can be performed using a computer.

‘Magnetic Resonance (MR) data’ as used herein encompasses the recorded measurements of radio frequency signals emitted by atomic spins by the antenna of a Magnetic resonance apparatus during a magnetic resonance imaging scan. Magnetic resonace data is an example of medical imaging data. A Magnetic Resonance Imaging (MRI) image is defined herein as being the reconstructed two or three dimensional visualization of anatomic data contained within the magnetic resonance imaging data. This visualization can be performed using a computer.

In an aspect of the invention the invention provides for a therapeutic apparatus. The therapeutic apparatus comprises a therapeutic system for treating a target zone of the subject. The therapeutic system has an adjustable focus directing radiation into the target zone. The therapeutic system essentially directs radiation into the target zone and has a means for focusing it. The therapeutic system may be for instance, but is not limited to, an x-ray therapy system, a charged particle therapy system, a proton therapy system, a high-intensity focused ultrasound system, a laser ablation system, and a cryo-ablation system.

The therapeutic apparatus further comprises a respiration sensor for measuring a respiratory phase of the subject. The data acquired with the respiration sensor is referred to as respiratory-phase-data. Data from the respiration sensor may be logged as a function of time. Respiratory-phase-data that is recorded as a function of time is referred to as time-dependent-respiratory-phase-data. A respiration sensor as used herein encompasses any sensor which is able to measure the respiratory phase of a subject. A respiration sensor is but is not limited to a breathing advisor or sensor, a force sensor, a sensor which measures the position or changes in position of the subject, an accelerometer, and fiduciary marks on the subject. The therapeutic apparatus further comprises a processor for controlling the therapeutic apparatus. It is understood herein that the processor is equivalent to a controller or a control system. Furthermore a processor may refer to multiple processors. The multiple processors may be within a single computer system or embedded system or the processors may be distributed amongst a collection or network of computers or embedded systems.

The therapeutic apparatus further comprises a memory containing machine executable instructions for execution by the processor. Execution of the instructions causes the processor to send control signals to the therapeutic system that cause treatment of the target zone. The control signals contain commands which cause the therapeutic system to generate a radiation source which treats the target zone locally. Execution of the instructions further causes the processor to receive time-dependent-respiratory-phase-data from the respiration sensor. Execution of the instructions further causes the processor to generate focus-adjustment-control-signals in accordance with the time-dependent-respiratory-data. The therapeutic system has an adjustable focus. The focus-adjustment-control-signals cause the focus of the therapeutic system to be adjusted. In other words the focus-adjustment-control-signals causes the focus with the location of the target zone within the subject to be shifted to a different location. Execution of the instructions further causes the processor to send the focus-adjustment-control-signals to the therapeutic system. When the therapeutic system receives the focus-adjustment-control-signals, these control signals causes the focus of the therapeutic system to be adjusted. The focus may be adjusted such that it compensates for motion of the target zone. This embodiment is advantageous because the therapeutic system may treat the target zone of the subject continuously. For instance, if the subject is undergoing respiration the anatomy of the subject may change as a function of time. By sending the focus-adjustment-control-signals to the therapeutic system the adjustable focus may be used to compensate for motion of the subject and the target zone may then move such that the same portion of the subject's anatomy or a desired portion of the subject's anatomy is treated.

In another embodiment, the instructions cause the processor to send control signals to the therapeutic apparatus which cause the therapeutic system to continuously treat the target zone while performing the steps of receiving the time-dependent-respiratory-phase-data, generating the focus-adjustment-control-signals-and sending the focus-adjustment-control signals to the therapeutic system.

In another embodiment the memory further contains a targeting model. A ‘targeting model’ as used herein encompasses a model which is descriptive of a location of the target zone as a function of the time-dependent-respiratory-phase-data. The focus-adjustment-control-signals are generated in accordance with the time-dependent-respiratory-phase-data and the targeting model. This embodiment is advantageous because a model is used which determines the location of the target zone within the subject. The time-dependent-respiratory-phase-data can be used to determine the location of the target zone. The model can then therefore be used to adjust the adjustable focus of the therapeutic system such that the target zone is treated as the subject is moving.

In another embodiment the therapeutic apparatus further comprises a medical imaging system for acquiring time-dependent-medical-image-data of the medical imaging zone. Time-dependent-medical-image-data is medical image data which is acquired and is a function of time. Execution of the instructions further causes the processor to receive the time-dependent-medical-image-data from the medical imaging system. Execution of the instructions further causes the processor to receive preliminary-time-dependent-respiratory-phase-data from the respiratory sensor. The time-dependent-medical-image-data and the preliminary-time-dependent-respiratory-phase-data are time correlated. The instructions further cause the processor to generate the targeting model in accordance with the time-dependent-medical-image-data and the preliminary-time-dependent-respiratory-phase-data. In this embodiment medical image data is acquired and used with respiratory-phase-data to make a model. This embodiment is advantageous because the model may be used with the later acquired time-dependent-respiratory-phase-data to predict the location of the target zone.

The use of the targeting model allows radiation to be directed into the target zone without the medical imaging system acquiring medical image data during a treatment of the target zone. For instance the model could be created using the medical imaging system and then the therapeutic system and the subject could be removed from the medical imaging system later and then the treatment of the target zone could be performed without the medical imaging system. This has the advantage that the medical imaging system is not needed during the entire treatment of the target zone. In the case of for instance magnetic resonance imaging where the medical imaging system is extremely expensive, this may reduce the expenses of the treatment.

In another embodiment the instructions cause the processor to generate the target model by registering time dependent locations of the target zone in the time-dependent-medical-image-data. This step may involve the reconstructing of the medical image data into a medical image. The process of registering locations in a medical image or medical data is well known in the art and involves pattern recognition or fitting a model to the image. The instructions further cause the processor to generate a target model by mapping a vector onto the registered time-dependent locations of the target zone as a function of the preliminary-time-dependent-respiratory-phase-data. In this step the time-dependent locations are mapped using a vector and the respiratory-phase-data. Using the later acquired time-dependent-respiratory-phase-data the location of a new vector may be calculated. This embodiment is advantageous because it provides an effective way of constructing a targeting model.

In another embodiment the instructions further cause the processor to compute a correlation between the preliminary-time-dependent-respiratory-phase-data and the time-dependent-respiratory-phase-data. The instructions further cause the processor to send stop control signals to the therapeutic system that cause the treatment of the target zone to stop if the computer correlation is below a predetermined correlation threshold. In this embodiment the respiratory-phase-data that was used to create the targeting model is compared to the respiratory-phase-data that is acquired during treatment of the target zone. If the correlation is below the predetermined correlation threshold this may indicate that the model will not accurately predict the location of the target zone within the subject. In this case the stop control signals cause the therapeutic system to stop treating the target zone. This is advantageous because if the subject is not following the same respiratory cycle or has moved or there is some other problem then the subject could be injured during treatment of the target zone. This embodiment provides a safety check to the system.

In another embodiment the medical imaging system is a magnetic resonance imaging system.

In another embodiment the medical imaging system is an ultrasound imaging system.

In another embodiment the medical imaging system is a computer tomography system.

In another embodiment the therapeutic apparatus further comprises a subject support for supporting the subject. The respiratory sensor is a force sensor integrated into the subject support such that the respiratory sensor supports at least a portion of the subject's weight. A force sensor as used herein is understood to be encompassed by a sensor which measures the force, a change in force, or the acceleration of a mass connected to the force sensor. This embodiment is advantageous because it is convenient to have the respiratory sensor integrated into a subject support. Additionally the use of a force sensor does not require the subject to be fitted with a special breathing tube or other measurement device.

In another embodiment the force sensor acquires force data. The force data comprises a ballistocardiogram signal. This embodiment is particularly advantageous because ballistocardiogram signal or data comprises data about the movement and also the respiratory cycle of a subject.

In another embodiment the therapeutic apparatus further comprises a low pass filter for filtering the force data into respiratory-phase-data. This embodiment is advantageous because the ballistocardiogram signal contains several different types of data. A low pass filter may be used to filter the force data into respiratory-phase-data. For instance the force sensor may be used to acquire the time-dependent-respiratory-phase-data and it may also be used to acquire the preliminary-time-dependent-respiratory-phase-data. The low pass filter may be implemented electronically or it may be implemented digitally in software or by a digital signal processing chip. For instance the force sensor could directly send the respiratory-phase-data to the processor, or in other embodiments the step of receiving the time-dependent-respiratory-phase-data could be raw data received from the force sensor. In this case a digital signal processing chip or machine executable instructions may cause the processor to digitally filter the force data to acquire the respiratory-phase-data.

In another embodiment the instructions further cause the processor to receive time-dependent force data from the force sensor. These instructions may be identical with those that cause the processor to receive time-dependent-respiratory-phase-data from the respiration sensor. The instructions further cause the processor to calculate an average energy signal in accordance with the time dependent force data. The instructions further cause the processor to send stop control signals to the therapeutic system that cause the treatment of the target zone to stop if the average energy is above a predetermined average energy threshold. This embodiment is advantageous because calculating an average energy signal for example by calculating the RMS or root mean square of the real-time time-dependent force data may indicate that the subject is under movement. If the subject has moved then the targeting model may no longer be valid. This embodiment therefore may provide a means of detecting when the targeting model is no longer valid.

In another embodiment the therapeutic system is a high-intensity focused ultrasound system integrated into the subject support. The high-intensity focused ultrasound system comprises an ultrasound transducer with an adjustable focus. The ultrasound transducer is therefore able to adjust ultrasound radiation or energy into the target zone. The adjustable focus may be focused via mechanical means through a mechanical positioning system which positions the location of the ultrasound transducer and/or the ultrasound transducer may focus the ultrasound energy electronically. For electronic focusing the ultrasound transducer may have multiple transducer elements on the surface of the ultrasound transducer. By controlling the energy and particularly the phase of ultrasound energy provided to each of the elements the focus may be adjusted. Combining the high-intensity focused ultrasound system with a force sensor is particularly advantageous. A subject may lie on the subject support thereby putting weight on both the high-intensity focused ultrasound system and the force sensor.

In another embodiment the therapeutic system is a gamma radiation treatment system.

In another embodiment the therapeutic system is a charge particle treatment system.

In another embodiment the therapeutic system is a therapeutic x-ray system.

In another embodiment the therapeutic system is a proton therapy system.

In another embodiment the therapeutic system is a cryo-ablation system. A cryo-ablation system as used herein encompasses a system which uses the freezing or the reduction of temperature of tissue to cause ablation.

In another embodiment the therapeutic system is a laser treatment or ablation system.

In another embodiment the therapeutic system is a radiation-frequency ablation or heating system.

In another embodiment the therapeutic system is a high-intensity focused ultrasound system.

In another embodiment the control signals cause the therapeutic system to cause treatment of the target zone while the processor is receiving the time-dependent-respiratory-phase-data from the respiration sensor, generating focus-adjustment-control-signals in accordance with the time-dependent respiratory data, and sending the focus-adjustment-control-signals to the therapeutic system.

This embodiment is particularly advantageous because in this embodiment the therapeutic system is treating the target zone while the respiratory-phase-data is received and while adjusting the focus-adjustment-control-signals. Essentially the therapeutic system is treating the target zone in a continuous fashion. This may lead to more rapid treatment and therapy than if the operation of the therapeutic system is simply gated by the time-dependent-respiratory-phase-data.

In another aspect the invention provides for a computer-implemented method of operating a therapeutic apparatus. The therapeutic apparatus comprises a therapeutic system for treating a target zone of the subject. The therapeutic system has an adjustable focus for directing radiation into the target zone. The therapeutic apparatus further comprises a respiration sensor for measuring a respiratory phase of the subject. The method comprises the step of sending control signals to the therapeutic system that cause treatment of the target zone. The method further comprises receiving time-dependent-respiratory-phase-data from the respiration sensor. The method further comprises the step of generating focus-adjustment-control-signals in accordance with the time-dependent-respiratory-phase-data. The method further comprises the step of sending the focus-adjustment-control-signals to the therapeutic system. The advantages of this method have been previously discussed.

In another aspect the invention provides for a computer program product comprising machine executable instructions for execution by a processor of a therapeutic apparatus. The computer program product may for instance be stored in a memory and may be a computer-readable storage medium. The therapeutic apparatus comprises a therapeutic system for treating the target zone of the subject. The therapeutic system has an adjustable focus for directing radiation into the target zone. The apparatus further comprises a respiration sensor for measuring a respiratory phase of the subject. Execution of the instructions cause the processor to send control signals to the therapeutic system that causes treatment of the target zone. Execution of the instructions further causes the processor to receive time-dependent-respiratory-phase-data from the respiration sensor. Execution of the instructions further causes the processor to generate focus-adjustment-control-signals in accordance with the time-dependent-respiratory-phase-data. Execution of the instructions further causes the processor to send the focus-adjustment-control-signals to the therapeutic system.

### BRIEF DESCRIPTION OF THE DRAWINGS

In the following preferred embodiments of the invention will be described, by way of example only, and with reference to the drawings in which:

FIG. 1 shows a flow diagram which illustrates a method according to an embodiment of the invention;

FIG. 2 shows a flow diagram which illustrates a method according to a further embodiment of the invention;

FIG. 3 illustrates a therapeutic apparatus according to an embodiment of the invention;

FIG. 4 illustrates a therapeutic apparatus according to a further embodiment of the invention;

FIG. 5 illustrates a therapeutic apparatus according to a further embodiment of the invention;

FIG. 6 illustrates a therapeutic apparatus according to a further embodiment of the invention;

FIG. 7a shows a plot of time dependent respiratory phase data;

FIG. 7b the location of the target zones 704 as a function of time

FIG. 7c vectors are assigned to the independent locations of the target zone 704.

FIG. 7d the use of the targeting model is illustrated.

FIG. 8 shows a further illustration of the force sensor

FIG. 9 shows time-dependent-respiratory-phase-data 900 that was acquired using a force sensor

FIG. 10 shows the root mean square of ballistocardiogram data

### DETAILED DESCRIPTION OF THE EMBODIMENTS

Like numbered elements in these figures are either equivalent elements or perform the same function. Elements which have been discussed previously will not necessarily be discussed in later figures if the function is equivalent.

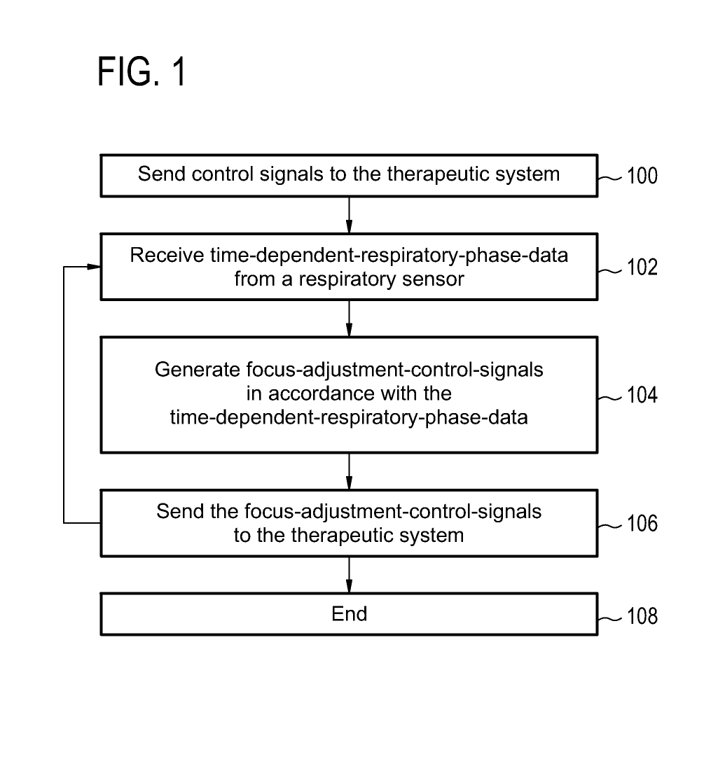

FIG. 1 shows a flow diagram which illustrates a method according to an embodiment of the invention. In step 100 control signals are sent to the therapeutic system. The control signals cause the therapeutic system to start treatment of the target zone of a subject. In step 102 time-dependent-respiratory-phase-data is received from a respiratory sensor. In step 104 the time-dependent-respiratory-phase-data is used to generate focus-adjustment-control-signals. In step 106 the focus-adjustment-control-signals are sent to the therapeutic system. The focus-adjustment-control-signals cause the therapeutic system to adjust its focus to a different location. Steps 102, 104, and 106 may be carried out repeatedly. This enables continuous treatment of the target zone. After the treatment of the target zone is complete, the method ends 108.

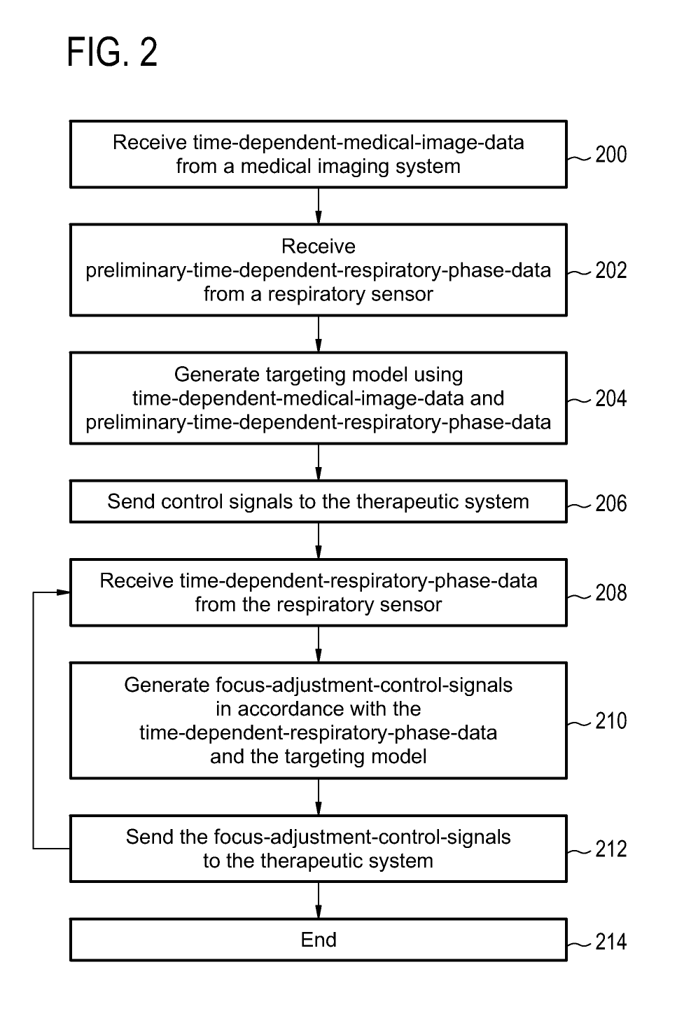

FIG. 2 shows a flow diagram which illustrates a method according to a further embodiment of the invention. In step 200 time-dependent-medical-image-data is received from a medical imaging system. In step 202 preliminary-time-dependent-respiratory-phase-data is received from a respiratory sensor. In step 204 a targeting model is generated using the time-dependent-medical-image-data and the preliminary-time-dependent-respiratory-phase-data. In step 206 control signals are sent to the therapeutic system. Step 206 of FIG. 2 is equivalent with step 100 of FIG. 1. In step 208 time-dependent-respiratory-phase-data is received from the respiratory sensor. Step 208 is equivalent to step 102 of FIG. 1. In step 210 focus-adjustment-control-signals are generated in accordance with the time-dependent-respiratory-phase-data and the targeting model. The targeting model which was generated in step 204 is used in step 210 to generate the focus-adjustment-control-signals. This may for instance be accomplished by using the targeting model to predict the location of the target zone and then generating focus-adjustment-control-signals which adjust the adjustable focus of the therapeutic system onto the target zone of the subject.

Finally in step 212 the focus-adjustment-control-signals are sent to the therapeutic system. Steps 208, 210 and 212 may be repeated many times, for instance during treatment of a target zone the respiratory-phase-data may be received continuously. This may cause the system to continuously generate focus-adjustment-control-signals and to continuously or periodically send them to the therapeutic system. When the target zone is complete step 214 is performed. In step 214 the treatment of the target zone is ended. This may be accomplished by sending control signals to the therapeutic system which cause the treatment of the target zone of the subject to cease.

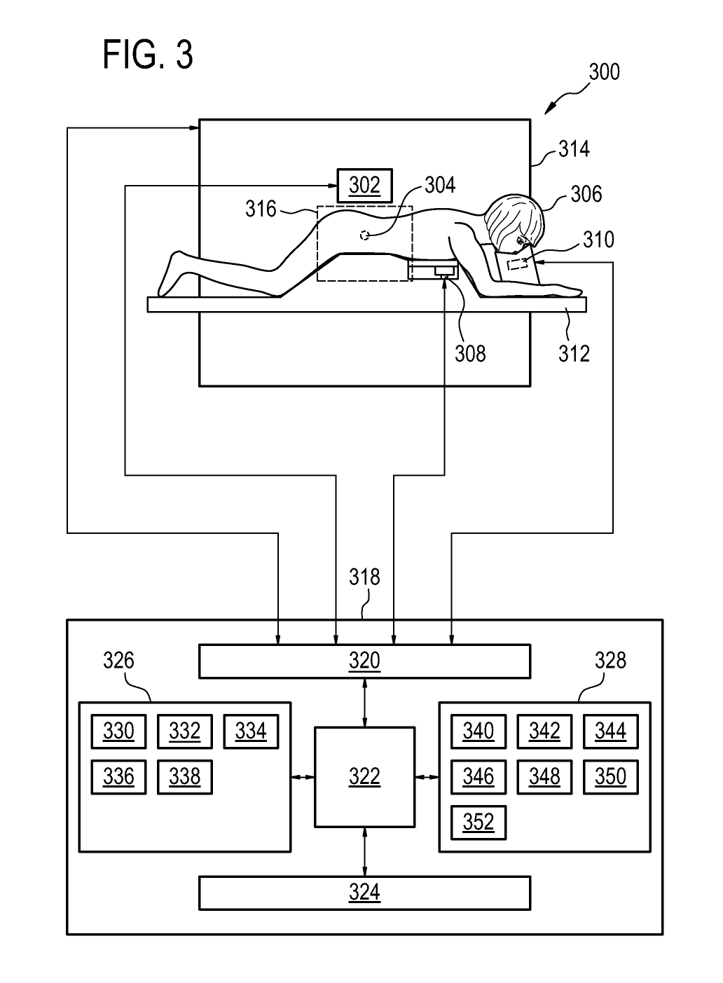

FIG. 3 shows a therapeutic apparatus 300 according to an embodiment of the invention. Shown is a therapeutic system 302 for treating a target zone 304 of a subject 306. The therapeutic system 302 is shown as a box and is representative of many different types of therapeutic systems. For example the therapeutic system could be but it not limited to: a gamma-radiation treatment system, a charged particle treatment system, a therapeutic or lineac x-ray therapy system, a proton therapy system, a cryo-ablation system, a laser treatment system, a radio-frequency ablation system, a radio-frequency heating system, and a high-intensity focused ultrasound system.

Shown in FIG. 3 are a first respiration sensor 308 and a second respiration sensor 310. The first respiration sensor 308 is representative of respiration sensors which track the motion or force exerted by a subject 306 as the subject breaths. The first respiration sensor could be but is not limited to: a force sensor, an accelerometer, a motion sensor, and a strain gauge.

Also shown in FIG. 3 is a second respiration sensor 310. The second respiration sensor 310 represents a class of respiration sensors that monitor the gas flow in or out of the subject 306 to determine a respiratory phase of the subject 306. For example the second respiration sensor could be, but is not limited to: a breathing advisor, a gas flow sensor, and an anesthesia respiration device.

Also shown in FIG. 3 is an optional medical imaging system 314. The medical imaging system 314 is for acquiring medical image data from an imaging zone 316. The subject 306 may be partially or completely within the medical imaging zone 316. The medical imaging system 314 acquires medical data which display anatomical or contains anatomical data of the subject 306 and may be used to identify the location of the target zone 304 as the subject breaths 306. As was mentioned before the medical imaging system 314 is optional in this embodiment. Elements which are also related to the medical imaging system such as control software for acquiring or processing medical image data are also optional. The therapeutic system 302, the first respiration sensor 308, the second respiration sensor 310, and the medical imaging system 314 are all shown as being connected to a hardware interface 320 of the computer system 318. The hardware interface 320 is connected to a processor 322 of the computer system 318. The hardware interface 320 allows a processor 322 to send and receive control signals to components of the therapeutic apparatus 300. The hardware interface 320 enables the processor 322 to control the therapeutic apparatus 300. The processor 322 is also shown as being connected to computer storage 326, computer memory 328, and a user interface 324.

The computer storage 326 is shown as containing time-dependent-respiratory-phase-data 330. This is respiratory-phase-data that was acquired using the first respiration sensor 308 and/or the second respiration sensor 310. For embodiments of the invention either or both of the first respiration sensor 308 and the second respiration sensor 310 may be present. Likewise the time-dependent-respiratory-phase-data 330 may be comprised of one or both of data from the first respiration sensor 308 and the second respiration sensor 310. The computer storage 326 is shown as further containing time-dependent-medical-image-data 332. The computer storage 326 is also shown as containing preliminary-time-dependent-respiratory-phase-data 334. The time-dependent-medical-image-data 332 and the preliminary-time-dependent-respiratory-phase-data 334 may be used to construct a target model 338. In some embodiments the time-dependent-medical-image-data 332 and the preliminary-time-dependent-respiratory-phase-data 334 may not be present. This may be because they have been deleted from storage or because a target model 338 may already exist. In some embodiments the time-dependent-medical-image-data has been reconstructed into time-dependent medical images 336 which are also located in the computer storage 326. The data shown in the computer storage 326 is representative if after the data has been used the data may not necessarily be retained within the computer storage 326 or computer memory 328. In some instances after a target model 338 is created the original data may be deleted.

The computer memory 328 is shown as containing a therapeutic apparatus control module 340. The therapeutic apparatus control module contains machine executable instructions which allow the processor 322 to operate and control the therapeutic apparatus 300. In some embodiments the computer memory 328 has a respiratory phase data analysis module 342. The respiratory phase data analysis module contains computer executable instructions for analyzing respiratory phase data. For example the respiratory phase data analysis module may contain code for digitally filtering the respiratory phase data 330, 334. The respiratory phase data analysis module 342 may also contain computer executable instructions for comparing the time-dependent-respiratory-phase-data 330 and the preliminary-time-dependent-respiratory-phase-data 334.

The computer memory 328 is shown as further containing a focus-adjustment-control-signal generation module 344. The focus-adjustment-control-signal generation module contains computer executable code that uses the time-dependent-respiratory-phase-data to generate focus-adjustment-control-signals. In some embodiments this is achieved using the target model 338. In some embodiments the computer memory 328 also contains a medical image reconstruction module 348. The medical image reconstruction module 348 contains computer executable code which allows the processor to reconstruct time-dependent-medical-image-data 332 into time dependent medical images 336.

In some embodiments the computer memory 328 also contains an image registration module 350. The image registration module contains computer executable code that is known in the art for performing image registration on the time dependent medical images 336. For instance the image registration module 350 may identify specific anatomical structures which allow identification of the target zone 304 as a function of time or respiratory phase. In some embodiments the computer memory 328 also contains a vector mapping module 352. The vector mapping module 352 may allow a target model generation module 346 to create a target model 338 using the preliminary-time-dependent-respiratory-phase-data 334 and registered time dependent medical images.

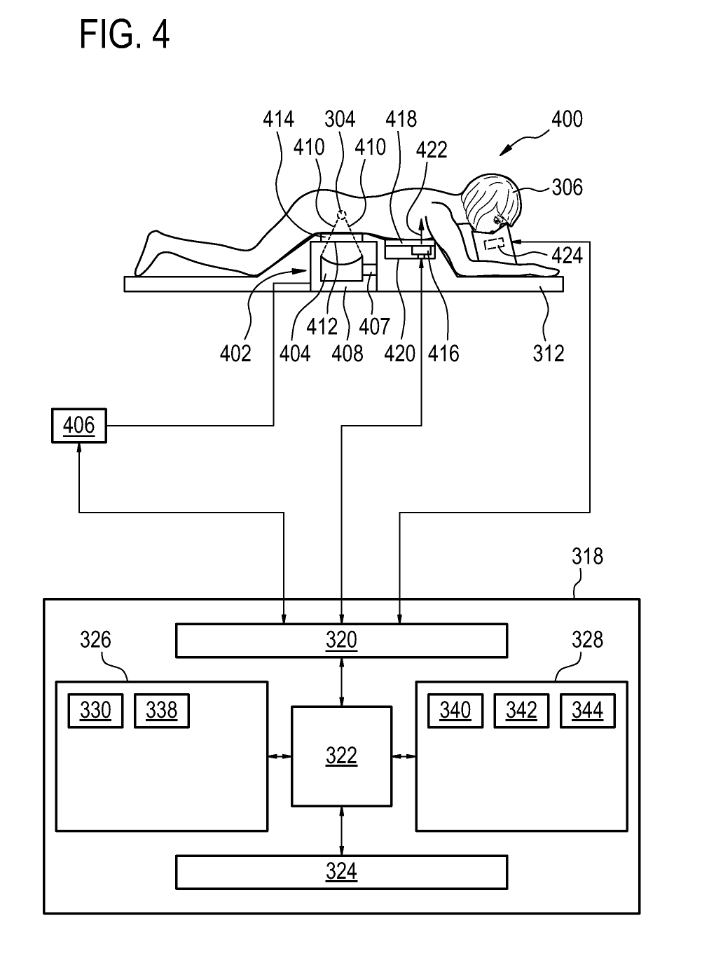

FIG. 4 shows a therapeutic apparatus 400 according to an alternative embodiment of the invention. This embodiment combines a high-intensity focused ultrasound system 402 and a force sensor 416 which are both incorporated into the subject support 312. The high-intensity focused ultrasound system 402 is integrated into the subject support 312. The high-intensity focused ultrasound system comprises an ultrasound transducer 404 which is connected to an ultrasound transducer power supply 406. The ultrasonic transducer 404 may contain multiple transducer elements. In this case the ultrasonic transducer power supply is able to control the amplitude and/or phase of electrical power supplied to the individual ultrasonic transducer elements. This allows electronic control of the focus of the ultrasound transducer 404. The ultrasound transducer 404 may also be positioned using a mechanical positioning system to mechanically move the ultrasonic transducer 404.

In this embodiment there are therefore two different ways of controlling the location of the focus of the ultrasonic transducer 404. The ultrasonic transducer 404 is shown as being immersed in a fluid-filled chamber 408. The fluid-filled chamber 408 is filled with a fluid which is able to conduct ultrasonic energy or waves from the ultrasonic transducer 404 to the ultrasonic window 412. The dashed lines 410 show the path of focused ultrasound to the target zone 304 located in the subject 306. The focused ultrasound 410 is shown as passing through the fluid-filled chamber 408 and passing through an ultrasound window 412. In this embodiment there is an optional gel pad 414 which couples the subject 306 to the ultrasound window 412 ultrasonically.

Also shown in FIG. 4 is a force sensor 416. The force sensor 416 is in contact with a mechanical adaptor 418 and a rigid support 420. The rigid support 420 is a support which connects the force sensor 416 to the subject support 312. The mechanical adaptor 418 provides a surface that the subject 306 can exert force onto. The mechanical adaptor 418 in turn exerts force on the force sensor 416. The force sensor 416 may measure the absolute force of the subject 306, a change in force, or it may measure the acceleration of the mechanical adaptor 418. The vector 422 is a counter force vector which is a force vector which balances the force exerted on the force sensor 416 by the mechanical adaptor 418.

Also shown in FIG. 4 is an optional breathing advisor 424. The breathing advisor 424 is also a respiratory sensor as is the force sensor 416. The respiratory advisor 424 may monitor the breathing of the subject 306 and provide supplementary data. The high-intensity focused ultrasound system 402, the force sensor 416 and the breathing advisor 424 are shown as being connected to the hardware interface 320 of a computer system 318. The computer system shown in FIG. 3 318 is equivalent to the computer system 318 shown in FIG. 4. The various components store data in the computer storage 326 and machine executable instructions stored in the computer memory 328 are also equivalent. What is noticeable in FIG. 4 is that many of the image analysis and data are absent from the computer storage 326 and computer memory 328. In this embodiment the target model 338 is pre-existing. This allows therapy to be performed on the subject 306 without the use of a medical imaging system 314 as was shown in FIG. 3.

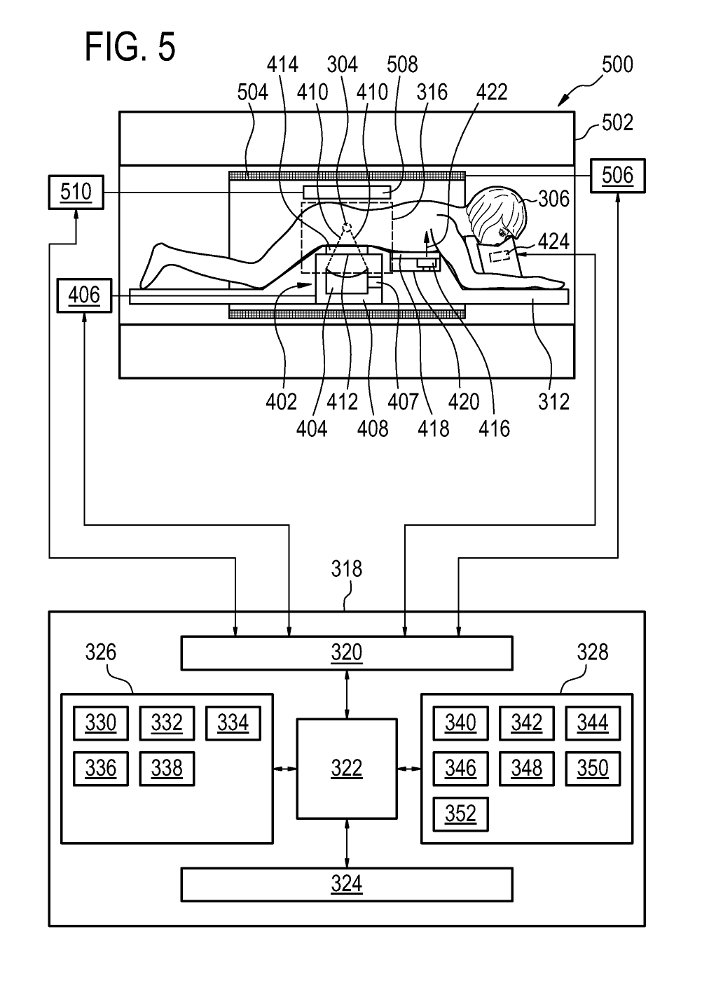

FIG. 5 shows a therapeutic apparatus 500 according to a further embodiment of the invention. The embodiment shown in FIG. 5 is equivalent to the embodiment shown in FIG. 4 except that a magnetic resonance imaging system is now incorporated into the therapeutic apparatus 500. Additional components which are not shown in FIG. 4 are discussed. In this embodiment there is a magnet 502 for generating a uniform magnetic field within an imaging zone 316. In this example a cross-sectional view of a cylindrical superconducting magnet is shown. Other magnet styles and types are known in the art and are also applicable to this invention. The subject support 312, high-intensity focused ultrasound system 402 and force sensor 416 are all shown as being within the bore of the cylindrical magnet 502. Also within the bore of the magnet is a magnetic field gradient coil 504. The magnetic field gradient coil 504 is actually three independent coil systems for spatially encoding magnetic spins within the imaging zone 316. Connected to the magnetic field gradient coil 504 is a magnetic field gradient coil power supply 506. The magnetic field gradient coil power supply 506 supplies current for energizing the magnetic field gradient coils 504.

Adjacent to the imaging zone 316 is a radio frequency coil 508. The radio frequency coil 508 is connected to a radio frequency transceiver 510. The radio frequency transceiver 510 is used for acquiring magnetic resonance data using the radio frequency coil 508. It is understood that the radio frequency coil 508 may represent individual transmit and receive coils. Likewise the radio frequency transceiver 510 also represents separate transmitters and receivers. The high-intensity focused ultrasound system 402, the force sensor 416, the optional breathing advisor 424, the magnetic field gradient coil power supply 506, and the radio frequency transceiver 510 are shown as being connected to a hardware interface 320 of a computer system 318. As with FIG. 4 the computer system 318 and the contents of the computer storage 326 and the computer memory 328 are equivalent with those shown in FIG. 3.

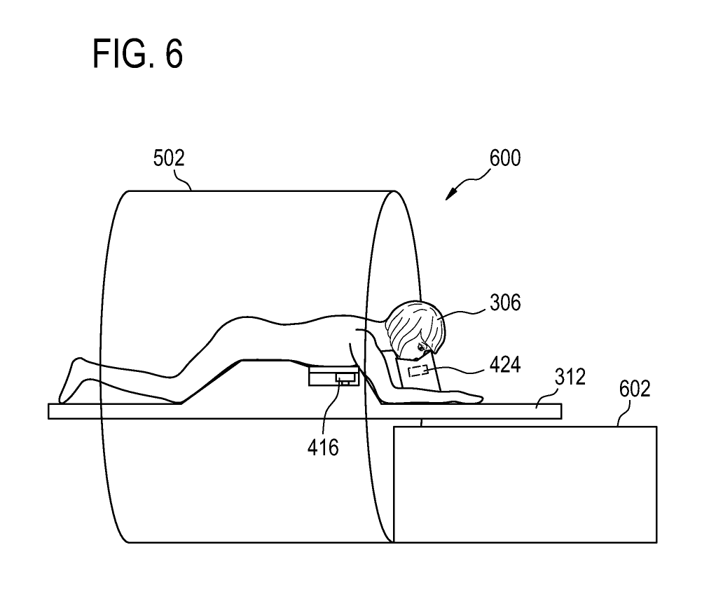

FIG. 6 shows an embodiment of a therapeutic apparatus 600 according to an embodiment of the invention. Not all details are illustrated in this Fig. This Fig. illustrates how a therapeutic apparatus 600 may be integrated into a conventional magnetic resonance imaging system. Shown in this Fig. is a cylindrical magnet 502. The subject support 312 with the force sensor 416 is placed into the magnet 502 using a trolley 602. A therapeutic system is not shown in this Fig. A high-intensity focused ultrasound system 402 as shown in FIGS. 4 and 5 nay be integrated into the subject support 312. Alternatively other types of therapeutic systems as were mentioned previously may also be integrated into the therapeutic apparatus 600 shown in this Fig. The trolley 602 may be used to insert or remove the subject support 312 into the magnet 502. This illustrates two things; first it illustrates how the invention may be integrated into a conventional magnetic resonance imaging system.

FIG. 6 also illustrates how the embodiment of FIG. 3 may be operated independent of an imaging system. For instance a subject support with the embodiment of a therapeutic apparatus 400 as shown in FIG. 4 may be inserted into the magnet 502. A target model 338 may be developed while the subject 306 and the subject support 312 are within the magnet 502 and the magnet resonance imaging system is operated. After the target model 338 has been developed the subject support 312 may be removed from the magnet 502. The treatment may proceed as is shown in the embodiment in FIG. 4. Now that the subject support 312 and the subject 306 have been removed from the magnet 502 the magnetic resonance imaging system may be used for other uses.

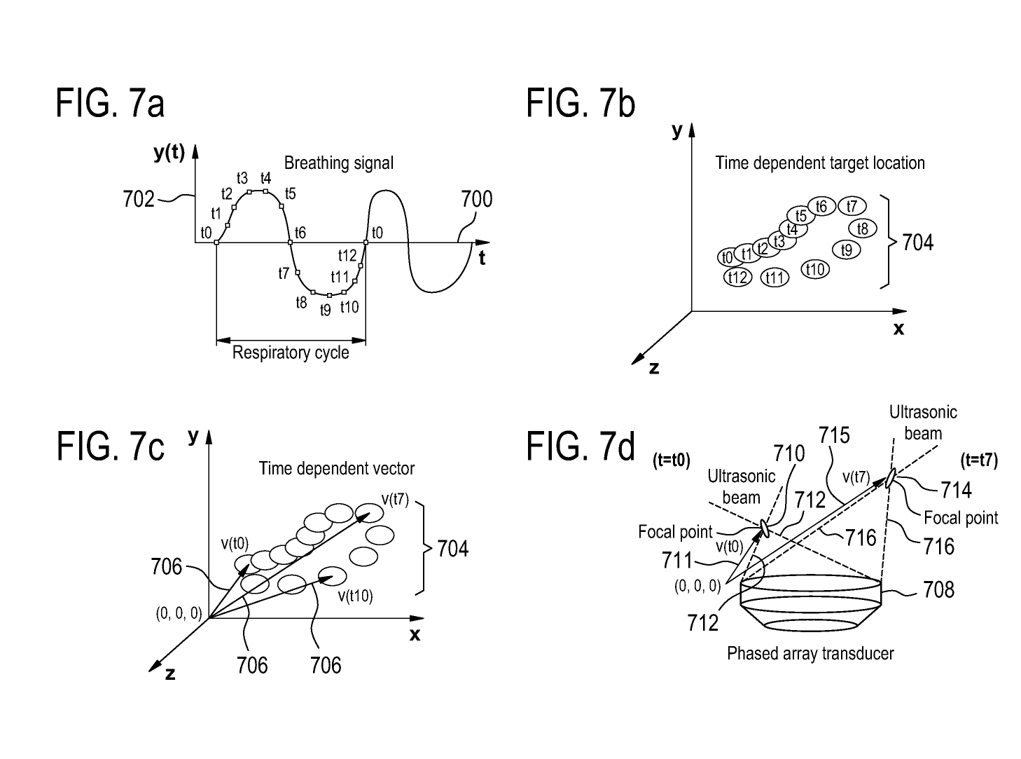

FIGS. 7a through 7d illustrate how a target model may be constructed and used. FIG. 7a has a time axis 700 and a time-dependent-respiratory-phase-data axis 702. In this plot data acquired from the respiration sensor is plotted as a function of time. The data points are labeled T0-T12. In FIG. 7b the location of the target zones 704 as a function of time is plotted in three-dimensional space. The location of the target zones is labeled T0-T12 also. In FIG. 7c vectors are assigned to the independent locations of the target zone 704. The assigning of vectors 706 to the target location 704 essentially creates the targeting model. Finally in FIG. 7d the use of the targeting model is illustrated. In this Fig. the treatment of a subject by an ultrasonic transducer 708 shown at various times. In this case the ultrasonic transducer 708 is a phased array transducer. The ultrasonic transducer is in other words constructed with multiple transducer elements. The Fig. also shows the location of the target zone at T0 710 and the location of the target zone at T7 714. The vector 711 identifies the predicted location of the target zone at T0. The vector 715 predicts the location of the target zone at T7. The dashed lines 712 show the path of focused ultrasound to the target zone 710 at time T0. The dashed line 716 shows the path of focused ultrasound to the target zone 714 at time T7. To determine the location of a target zone at times intermediate to when vectors have been created the intermediate vector could be calculated by interpolating between the two vectors.

Such a targeting model as is illustrated in FIG. 7 may compensates for periodic respiratory movement which is constant enough to obtain target location in reliable manner. Suggested steps to ensure constant breathing during the pre-treatment and treatment sessions are as follows:
1. Respiratory measurement may be started immediately after the patient is positioned on the table top.
2. The respiratory signal may be analyzed and the system informs the operator when the patient respiration is settled.
3. A “normal” respiratory signal may be generated and stored for the current treatment session. The “normal” signal can be a set of parameters, fitted curve or other model.
4. The respiration may be monitored during the treatment and it is constantly compared to the “normal” respiration to be sure that the target is moving in similar manner in different phases of the respiratory cycle.
5. If too low correlation is detected, the sonication is paused and the patient may be advised to recover normal breathing with a visual breathing advisor or other method. If the patient is unable to recover normal breathing, the target movement analysis is performed again before the sonication is allowed to continue.

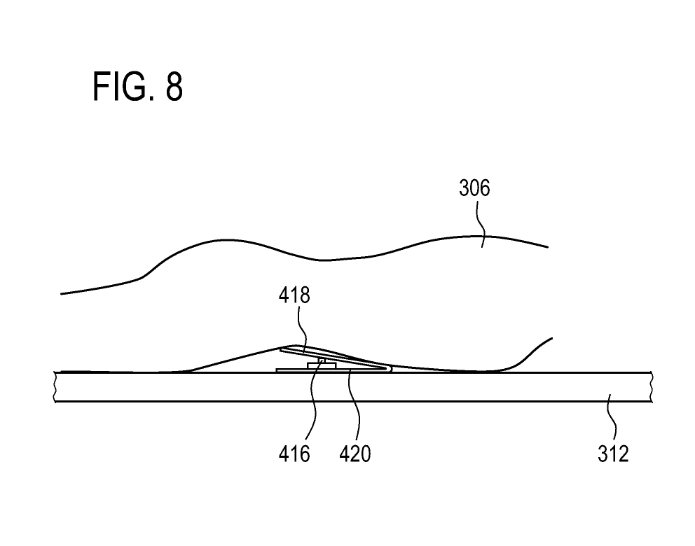

FIG. 8 shows a further illustration of the force sensor 416 that was previously described in the embodiments shown in FIGS. 3 and 4. In this embodiment there is a subject support 312 for supporting a subject 306. There is a mechanical adaptor 418 which is in contact with the subject 306 and the force sensor 416. The subject 306 exerts force on the mechanical adaptor 418. As the subject breaths 306 the force exerted on the mechanical adaptor 418 changes. The mechanical adaptor 418 then transmits force to the force sensor 416. The force sensor 416 is supported between the mechanical adaptor and the rigid support 420. The rigid support 420 transmits force to the subject support 312.

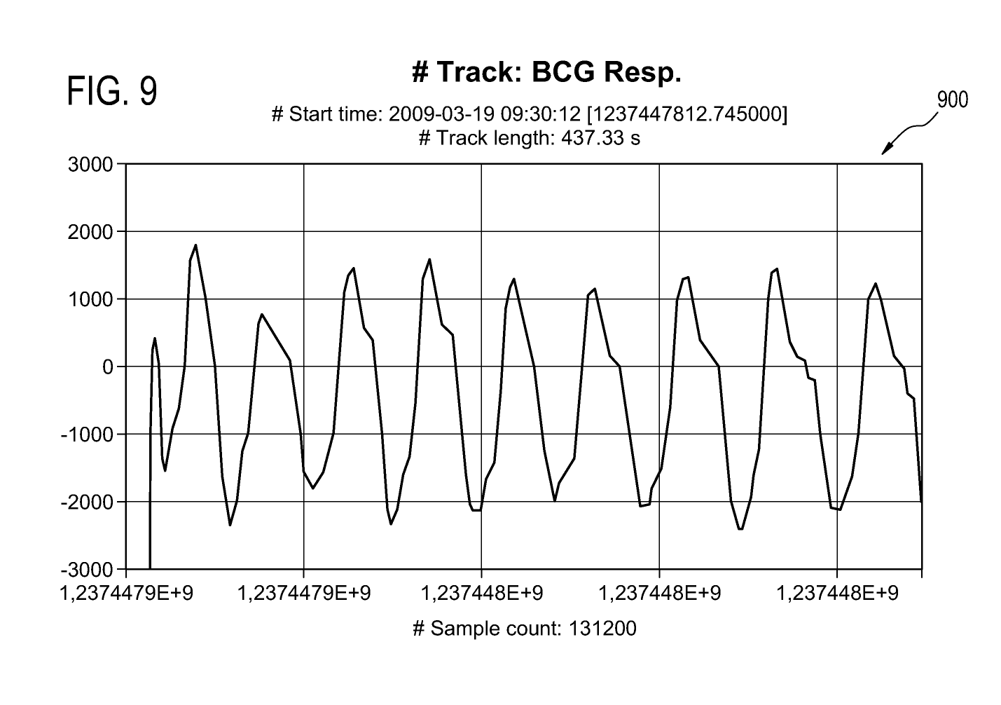

FIG. 9 shows time-dependent-respiratory-phase-data 900 that was acquired using a force sensor as illustrated in FIGS. 4, 5, and 8. The data shown in FIG. 9 is respiratory phase data acquired using a ballistocardiogram signal that was put through a low pass filter. Data was acquired in a 3 Tesla magnetic field using the arrangement shown in FIG. 8.

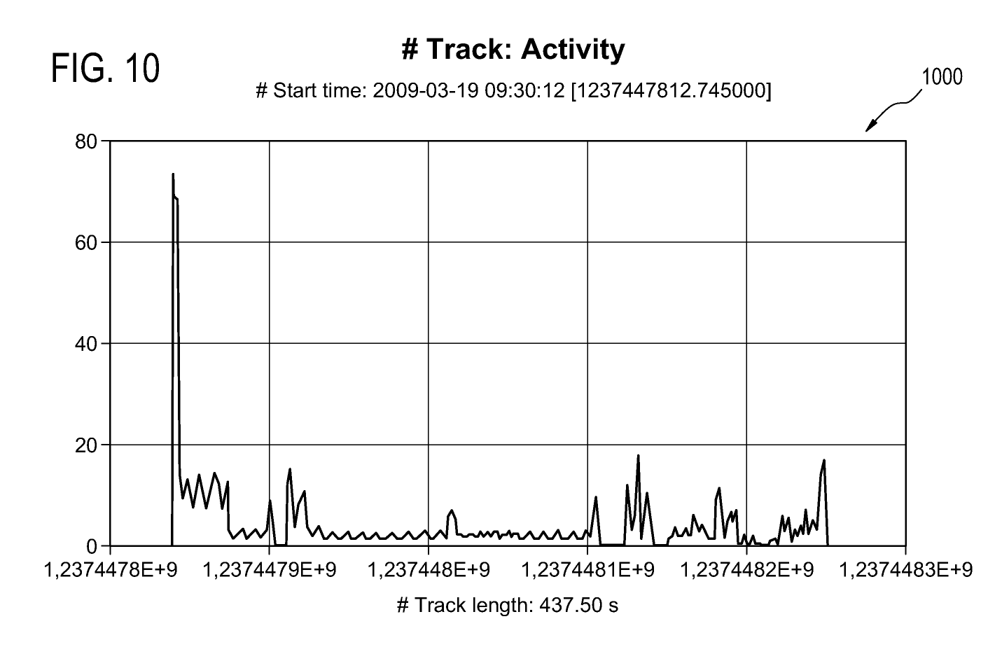

FIG. 10 shows ballistocardiogram data as was acquired in FIG. 9 but in this case the root mean square of the signal was calculated. This is an average energy signal 1000. The signal shown in FIG. 10 may be used to detect gross motion or movement by the subject. By placing a sufficient threshold or thresholds on data acquired in this way the motion of a subject may be detected and used to halt treatment of the target zone. Data was acquired in a 3 Tesla magnetic field using the arrangement shown in FIG. 8.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

### LIST OF REFERENCE NUMERALS

300 therapeutic apparatus

302 therapeutic system

304 target zone

306 subject

308 first respiration sensor

310 second respiration sensor

312 subject support

314 medical imaging system

316 imaging zone

318 computer system

320 hardware interface

322 processor

324 user interface

326 storage

328 memory

330 time-dependent-respiratory-phase-data

332 time-dependent-medical-image-data

334 preliminary-time-dependent-respiratory-phase-data

336 time dependent medical images

338 target model

340 therapeutic apparatus control module

342 respiratory phase data analysis module

344 focus-adjustment-control-signal generation module

346 target model generation module

348 medical image reconstruction module

350 image registration module

352 vector mapping module

400 therapeutic apparatus

402 high intensity focused ultrasound system

404 ultrasonic transducer

406 ultrasonic transducer power supply

407 mechanical positioning system

408 fluid filled chamber

410 path of ultrasound

412 ultrasound window

414 gel pad

416 force sensor

418 mechanical adaptor

420 rigid support

422 counter force vector

424 breathing advisor

500 therapeutic apparatus

502 magnet

504 magnetic field gradient coil

506 magnetic field gradient coil power supply

508 radio-frequency coil

510 radio-frequency transceiver

600 therapeutic apparatus

602 trolley

700 time axis

702 time-dependent-respiratory-phase-data axis

704 location of target zone

706 vectors mapping location of target zones for different respiratory phases

708 ultrasonic transducer

710 location of target zone at t₀

711 vector for time t₀

712 path of focused ultrasound at t₀

714 location of target zone at t₇

715 vector for time t₇

716 path of focused ultrasound at t₇

900 time-dependent-respiratory-phase-data

1000 averaged energy signal

1. A therapeutic apparatus comprising:
a therapeutic system for treating a target zone of a subject, wherein the therapeutic system has an adjustable focus for directing radiation into the target zone;
a respiration sensor for measuring a respiratory phase of the subject;
a processor for controlling the therapeutic apparatus; and
a memory containing machine executable instructions for execution by the processor, wherein execution of the instructions causes the processor to:
send control signals to the therapeutic system that cause treatment of the target zone,
receive time-dependent-respiratory-phase-data from the respiration sensor,
generate focus-adjustment-control-signals in accordance with the time-dependent-respiratory-phase-data, and
send the focus-adjustment-control-signals to the therapeutic system.

2. The therapeutic apparatus of claim 1, wherein the memory further contains a targeting model, wherein the targeting model is descriptive of a location of the target zone as a function of the time-dependent-respiratory-phase-data, and wherein the focus-adjustment control signals are generated in accordance with the time-dependent-respiratory-phase-data and the targeting model.

3. The therapeutic apparatus of claim 2, further comprising a medical imaging system structured and configured for acquiring time-dependent-medical-image-data of a medical imaging zone, wherein execution of the instructions further causes the processor to:
receive the time-dependent-medical-image-data from the medical imaging system;
receive preliminary-time-dependent-respiratory-phase-data from the respiratory sensor, wherein the time-dependent-medical-image-data and the preliminary-time-dependent respiratory-phase-data are time correlated; and
generate targeting model in accordance with the time-dependent-medical-image-data and the preliminary-time-dependent-respiratory-phase-data.

4. The therapeutic apparatus of claim 3, wherein execution of the instructions cause the processor to generate the target model by:
registering time dependent locations of the target zone in the time-dependent-medical-image-data, and
mapping a vector onto the registered time dependent locations of the target zone as a function of the preliminary-time-dependent-respiratory-phase-data.

5. The therapeutic apparatus of claim 3, wherein execution of the instructions further cause the processor to:
compute a correlation between the preliminary-time-dependent-respiratory-phase-data and the time-dependent-respiratory-phase-data; and
send stop control signals to the therapeutic system that cause the treatment of the target zone to stop if the computed correlation is below a predetermined correlation threshold.

6. The therapeutic apparatus of claim 3, wherein the medical imaging system comprises at least one of the following: a magnetic resonance imaging system, an ultrasound imaging system, or a computed tomography system.

7. The therapeutic apparatus of claim 1, further comprising a subject support for supporting the subject, wherein the respiratory sensor is a force sensor integrated into the subject support such that the respiratory sensor supports at least a portion of the subject's weight.

8. The therapeutic apparatus of claim 7, wherein the force sensor is structured and configured to acquires time-dependent-force-data, and wherein the time-dependent-force-data comprises a ballistocardiogram signal.

9. The therapeutic apparatus of claim 7 further comprising a low pass filter structured and configured for filtering the force data into respiratory-phase-data.

10. The therapeutic apparatus of claim 6, wherein execution of the instructions further cause the processor to:
receive time dependent force data from the force sensor;
calculate an averaged energy signal in accordance with the time dependent force data; and
send at least one stop control signals to the therapeutic system that cause the treatment of the target zone to stop if the averaged energy signal is above a predetermined averaged energy threshold.

11. The therapeutic apparatus of claim 7, wherein the therapeutic system comprises a high intensity focused ultrasound system integrated into the subject support, wherein the high intensity focused ultrasound system comprises an ultrasound transducer with an adjustable focus.

12. The therapeutic apparatus of claim 1, wherein the therapeutic system comprise at least one of the following: a gamma-radiation treatment system, a charged particle treatment system, a therapeutic X-ray system, a proton therapy system, a cryo-ablation system, a laser treatment system, a radio-frequency ablation system, or a high intensity focused ultrasound system.

13. The therapeutic apparatus of claim 1, wherein the control signals cause the therapeutic system to cause treatment of the target zone while the processor is:
receiving the time-dependent-respiratory-phase-data from the respiration sensor,
generating the focus-adjustment-control-signals in accordance with the time dependent respiratory data, and
sending the focus-adjustment-control-signals to the therapeutic system.

14. A computer-implemented method of operating a therapeutic apparatus, wherein the therapeutic apparatus comprises a therapeutic system for treating a target zone of a subject, wherein the therapeutic system has an adjustable focus for directing radiation into the target zone, wherein the therapeutic apparatus further comprises a respiration sensor for measuring a respiratory phase of the subject, wherein the method comprises the steps of:
sending control signals to the therapeutic system that cause treatment of the target zone,
receiving time-dependent-respiratory-phase-data from the respiration sensor,
generating focus-adjustment-control-signals in accordance with the time-dependent-respiratory-phase-data, and
sending the focus-adjustment-control-signals to the therapeutic system.

15. A computer program product comprising machine executable instructions for execution by a processor of a therapeutic apparatus, wherein the therapeutic apparatus comprises a therapeutic system for treating a target zone of a subject, wherein the therapeutic system has an adjustable focus for directing radiation into the target zone, wherein the apparatus further comprises a respiration sensor for measuring a respiratory phase of the subject, wherein execution of the instructions cause the processor to:
send control signals to the therapeutic system that cause treatment of the target zone,
receive time-dependent-respiratory-phase-data from the respiration sensor,
generate focus-adjustment-control-signals in accordance with the time dependent respiratory data, and
send the focus-adjustment-control-signals to the therapeutic system.
